(19) United States
(12) Patent Application Publication
(10) Pub. No.: US 2026/0273267 A1
(43) Pub. Date: Sep. 17, 2026

(54) NONINVASIVE WIRELESS EAR-CLIP NEUROSTIMULATOR

(71) Applicant: COGNIGUARD MEDICAL HOLDINGS LIMITED, Nicosia (CY)

(72) Inventors: Tamir Ben David, Tel Aviv (IL); Raanan Gefen, Re'ut (IL)

(73) Assignee: COGNIGUARD MEDICAL HOLDINGS LIMITED, Nicosia (CY)

(21) Appl. No.: 19/516,187

(22) PCT Filed: Sep. 3, 2024
(86) PCT No.: PCT/IL2024/050884

§ 371 Date: Mar. 4, 2026

Related U.S. Application Data
(60) Provisional application No. 63/580,373, filed on Sep. 3, 2023.

(51) Int. Cl. A61N 1/04; A61N 1/36

(52) U.S. Cl. CPC A61N 1/0472; A61N 1/0456; A61N 1/36025; A61N 1/36031; A61N 1/36036

(57) ABSTRACT

Disclosed herein is a noninvasive auricular neurostimulator comprising a stimulation device for activating an auricular branch of the Vagus nerve, said stimulation device comprising an active electrode facing a concha cavum of the ear, and a return electrode facing an Eminence of the concha, or the retro auricular sulcus.

### FIELD

Disclosed herein is a noninvasive wireless ear-clip neurostimulator in the field of noninvasive neurostimulation.

### BACKGROUND

Neurostimulation facilitates enhancing neurological abilities of individuals suffering from neurological disorders or for cognitive enhancements.

FIGS. 1A-1C show a human ear 10. FIGS. 1A-1B show a front of ear 10 and Fig. C shows the rear of ear 10. FIG. 1A shows nerve geography of ear 10, which are auriculotemporal nerve 1, lesser occipital nerve 2, greater auricular nerve 3 and auricular branch of vagus nerve 5. FIG. 1B shows a concha 11 of ear 10. The auricular branch of the Vagus nerve is found in Concha 11. FIG. 1C shows sections on rear of 10 such as Eminence of Concha 30, Retro Auricular Sulcus 31 and Sulcus of Crux Antihelix 32.

### SUMMARY

The following embodiments and aspects thereof are described and illustrated in conjunction with systems, tools and methods which are meant to be exemplary and illustrative, not limiting in scope.

There is provided, in accordance with an embodiment, a noninvasive auricular neurostimulator including a stimulation device for activating an auricular branch of the Vagus nerve, said stimulation device including an active electrode facing a concha cavum of the ear; and, a return electrode facing an Eminence of the concha, or the retro auricular sulcus.

In some embodiments, the noninvasive auricular neurostimulator further includes an elastic clip.

In some embodiments, said elastic clip comprises requires pressure power within a range of 10 to 500 milliBar.

In some embodiments, the noninvasive auricular neurostimulator further includes a communication system.

In some embodiments, said communication system is wireless.

In some embodiments, said return electrode comprises an active area within a range of 100 to 1000 squared millimeters.

In some embodiments, said active electrode comprises an active area within a range of 50 to 400 squared millimeters.

In some embodiments, said active electrode and said return electrode are disposable in part or in full.

In some embodiments, said active electrode and said return electrode each comprise a disposable electrically conductive gel leaf.

In some embodiments, a stimulation activity is synchronized to the physiological states of a patient.

In some embodiments, said stimulation device is configured to treat neurological disorders.

In some embodiments, the neurological disorder is Alzheimer disease.

In some embodiments, the noninvasive auricular neurostimulator further includes two stimulation sites, one site on the left ear concha and the other site on the right ear concha.

In some embodiments, the two stimulation sites are connected by an elastic bar that provides pressure at an electrode interface within the concha.

In some embodiments, the one or more electrode on one or 2 ears are configured for sensing Electroencephalography (“EEG”) or Electrocardiogram (ECG) signals.

In some embodiments, the noninvasive auricular neurostimulator further includes an additional module configured for acoustic stimulation or mechanical vibration stimulation.

In some embodiments, the noninvasive auricular neurostimulator further includes an additional headband configured to directly charge said auricular neurostimulator device.

In some embodiments, said electrodes are placed in a way that the electrodes are pushed toward the concha skin or having an adhesive gel that enables contact with the skin.

In some embodiments, said stimulation device is positioned adjacent to the ear and connected with cables to said active electrode.

In some embodiments, the noninvasive auricular neurostimulator further includes a wireless charger that provides wireless charging from a headband to said stimulation device, wherein said wireless charger is positioned adjacent to said device.

In some embodiments, said electrodes comprise a cooling element to cool the stimulation site.

In some embodiments, said stimulation device further comprises a position sensor configured to detect sides sleep or back sleep postures.

In some embodiments, the noninvasive auricular neurostimulator further includes a photovoltaic charging surface used for charging said stimulation device.

In addition to the exemplary aspects and embodiments described above, further aspects and embodiments will become apparent by reference to the figures and by study of the following detailed description.

### BRIEF DESCRIPTION OF THE FIGURES

Some non-limiting exemplary embodiments or features of the disclosed subject matter are illustrated in the following drawings.

References to previously presented elements are implied without necessarily further citing the drawing or description in which they appear.

FIGS. 1A-1C are prior art illustrations of the nerve geography of a human ear;

FIG. 2 is a schematic illustration of a noninvasive miniature neurostimulator system, according to certain exemplary embodiments;

FIGS. 3A-3E are schematic illustrations of an ear clip neurostimulator structure mounting onto an ear, according to certain exemplary embodiments;

FIG. 4A is a schematic illustration of an active electrode, according to certain exemplary embodiments;

FIG. 4B is a schematic illustration of a return electrode, according to certain exemplary embodiments; and

FIG. 5 shows a dual ear neurostimulator structure and mounting, according to certain exemplary embodiments.

Identical, duplicate, equivalent or similar structures, elements, or parts that appear in one or more drawings are generally labeled with the same reference numeral, optionally with an additional letter or letters to distinguish between similar entities or variants of entities, and may not be repeatedly labeled and/or described. Dimensions of components and features shown in the figures are chosen for convenience or clarity of presentation and are not necessarily shown to scale or true perspective. For convenience or clarity, some elements or structures are not shown or shown only partially and/or with different perspective or from different points of view.

### DETAILED DESCRIPTION

Described herein is a noninvasive wireless ear-clip neurostimulator, according to certain exemplary embodiments.

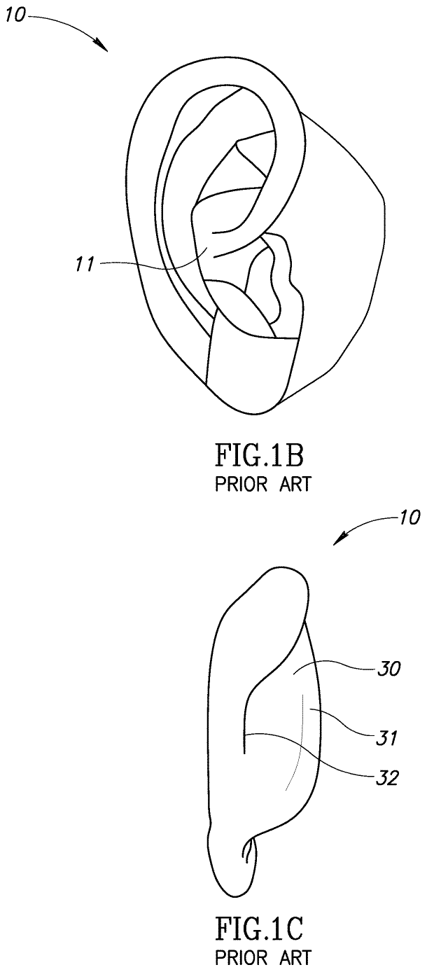

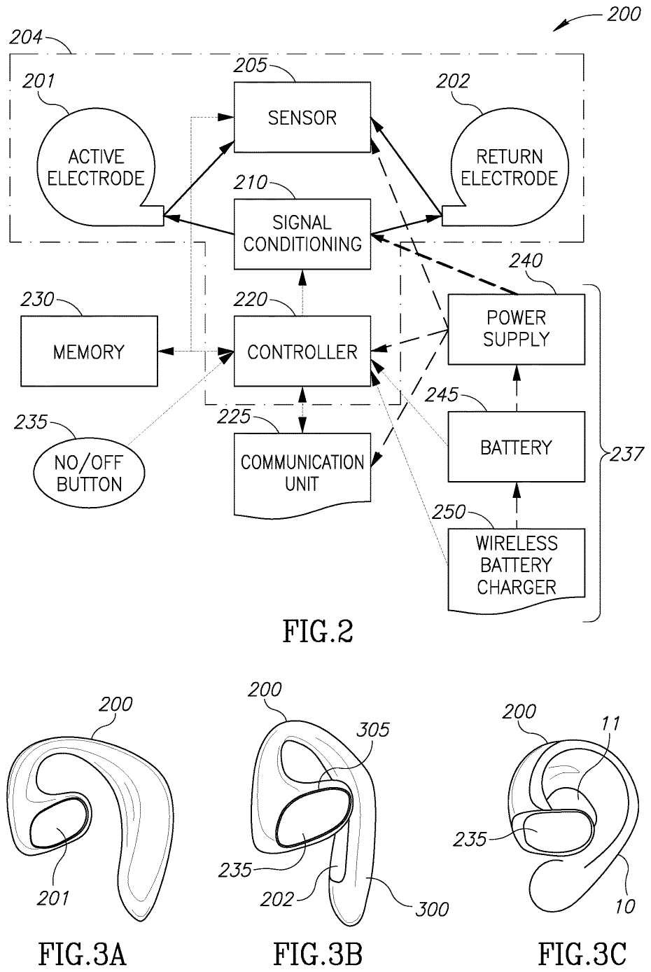

With reference to FIG. 2, which is a schematic illustration of a noninvasive miniature neurostimulator system (“system”) 200 including four interconnected subsystems that work together to achieve maximum performance with the least amount of space and power: powering, stimulation, sensing, and communication, according to certain exemplary embodiments. System 200 is installed within the structure of ear clip neurostimulator structure 300 (FIGS. 3A-3E) to facilitate achieving the desired stimulation disclosed herein.

System 200 includes a power subsystem that can be a rechargeable battery 245, such as a Lithium-Ion (“Li-Ion battery”), with a wired or wireless battery charger 250 in charge of conditioning battery charging process and receiving external power. Power subsystem 237 includes a power supply circuitry 240 positioned at a connective end of a battery 245, thereby configured to draw power from battery 245 and convert the power as needed to high voltages of at least 10V and up to 40V for operation of a stimulation circuitry and low voltages of 3V or 5V for the controlling circuitries such as a controller 220, sensor 205 operation, and communication unit 225 operation.

In some embodiments, the powering subsystem 237 operates in conjunction with other components of system 200 to improve power supply and increase the efficiency of system 200 in addition to providing electricity to all components of system 200. During stimulation, controller 220 rapidly activates the high voltage circuitry. Controller 220 is configured to establish the energy charging status of battery 245 and transmits a report via a communication unit 225, for example, via a Bluetooth link. When low power is detected, controller 220 can switch system 200 into sleep or a Hibernation mode, which disables stimulation and protects memory 230. When controller 220 detects charging activity by the power subsystem, controller 220 activates communication unit 225 and configures it to be available for communication pairing with external devices and for performing other maintenance tasks.

In some embodiments, Communication unit 225 can execute a Bluetooth (BT) model, or other close range communication model, which communicates with an external controller to send and receive messages through a secure protocol.

The controller 220 can receive incoming messages via communication unit 225 and provide the messages to set the system parameters, which are then stored in memory 230. For example, the incoming messages can include ON and OFF commands that instruct controller 220 how to execute stimulation. In some embodiments, system 200 can include an ON/OFF button 235 which is configured as an input to switch the system to “On” and “Off” states.

System 100, through communication unit 225, can send outgoing messages that can include reports of various system parameters to an external device, such as battery charging status, impedance parameters as determined by a sensor, the system settings as stored in memory 230, an activity summary of predefined duration, such as nighttime, or the like. In some embodiments, the necessary data to report these metrics is stored by controller 220 in memory 230. Controller 225 is configured to generate and provide the message to communication unit 225, which transmits the message according to instructions provided by controller 225.

A sensing subsystem 204 is operated by controller 220 to facilitate monitoring the separation of stimulation and sensing, such that that no stimulation is carried out during a sensing process. One or more sensors 205 provide a controlled low voltage and low current signal through system electrodes—active electrode 201 and return electrode 202, and an output circuitry, which changes according to the skin impedance characteristics, which can be measured by changes in the voltage. Controller 220 subsequently converts the measured change in voltage to impedance measurements.

Ohm's equation,

$$R = \frac{V}{I} ,$$

in which “V” is the voltage and “I” is the current and “R” is the resistance, which is a component of the impedance that can be derived. The relevant voltage and current for the resistance calculation are measured at the beginning of the power pulse due to the capacitance behavior of the skin impedance.

The equation,

$$C = \frac{q}{V} ,$$

in which “V” is the voltage, “q” is the charge, and “C” is the capacitance component of the impedance. The current can be aggregated over time to approximate the charge. In some embodiments, implementation of a stimulator system that delivers a constant electrical current over a set period of time can produce an effective change in potential:

$$C = \frac{I}{( \frac{dV}{dt} )} .$$

Therefore, detecting the potential variation across the pulse duration will enable accurate detection of the capacitance component of the impedance.

The stimulation subsystem 204 is the primary function of the system 200 and consist of the controller 220 which administers the activity of system 200 and signal conditioning circuit 210 which delivers the high voltage stimulation to the active electrode 201 while using the return electrode 202 as grounding.

The stimulation can be administered in short bursts of within a range of 50 to 500 or 500 to 5,000 microseconds, with the current being controlled to a predefined level of 0.1 to 5 micro Amps (“mA”). The pulses can be sent positively by active electrode 201 (i.e., the positive pole) or negatively (i.e., the positive pole) is delivered by return electrode 202. In some embodiments, a similar pulse with the opposite sign, for example, a negative instead of positive, can be delivered after the stimulation pulse to balance the charge. There should always be enough time between stimulation pulses where both electrodes-active electrode 201 and electrode 202, are grounded, for example, more than 5 or 10 or 15 time constants, to prevent charge accumulation and to allow any leftover charges to dissipate.

During the active duration of the stimulation, which can last 5 to 60 minutes, often 30 minutes, the stimulation pulses are administered at a rate in the range of 1 to 100 Hz or 10 to 600 Hz, commonly 25 Hz. A washout period of 5 to 60 minutes will be applied after each active period, with 30 minutes being the norm during which no stimulation will take place. In some applications, such as sleep stages or external visual-auditory stimulation, the active and washout durations synchronize to physiological activity of the ear 10.

In some embodiments, as part of the setting message, the external device can define the stimulation parameters within a designed range. A technician may have access to all changing all of the stimulation parameters through the remote device. However, in some cases, so as to focus on the treatment process, only the stimulation current is changed during the treatment. To guarantee safe and unnoticed stimulation, the stimulation current may be adjusted for each patient to a level within a range of 0.1 to 0.3 mA below the level at which the patient feels the stimulation.

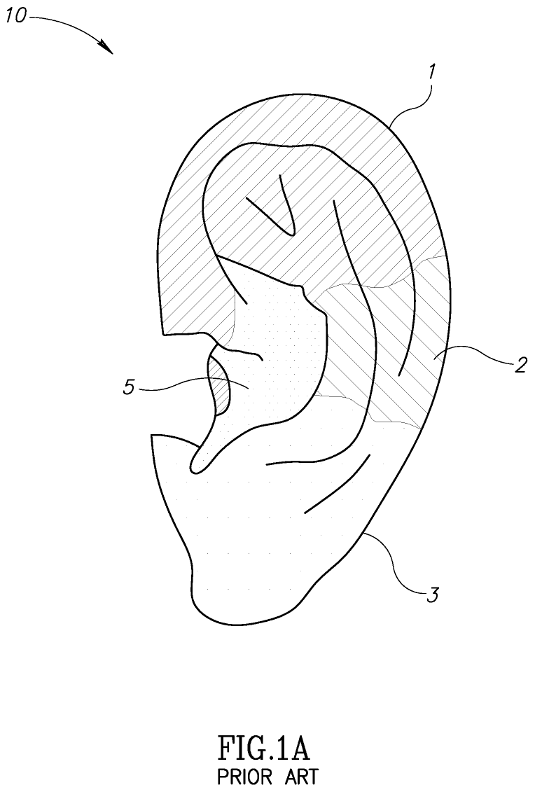

With reference to FIG. 3A-3E, which are schematic illustrations of an ear clip neurostimulator structure (“ear clip”) 300 mounting onto an ear 10 (FIGS. 1A-1C), according to certain exemplary embodiments. A front section of ear clip 300 is configured to fit over the Concha cavum 11 and a back section of ear clip 300 is configured to be mounted over the Eminence of the Concha 11. The Concha's auricular branch of the Vagus nerve, which is locally stimulated, is the basis of the effectiveness of system 100. The active electrode 201, which is positioned over the concha cavum, transmits the stimulation to the return electrode 202, which is located at the Eminence of the Concha on the other side of the ear.

A sound canal that allows sound to enter the ear canal can also be seen on the front half of ear clip 300, along with On/Off button 235 for versatile activation. Ear clip 300 provides a house structure for the electrical components of system 200 (FIG. 2), providing enough space without the need for external wiring.

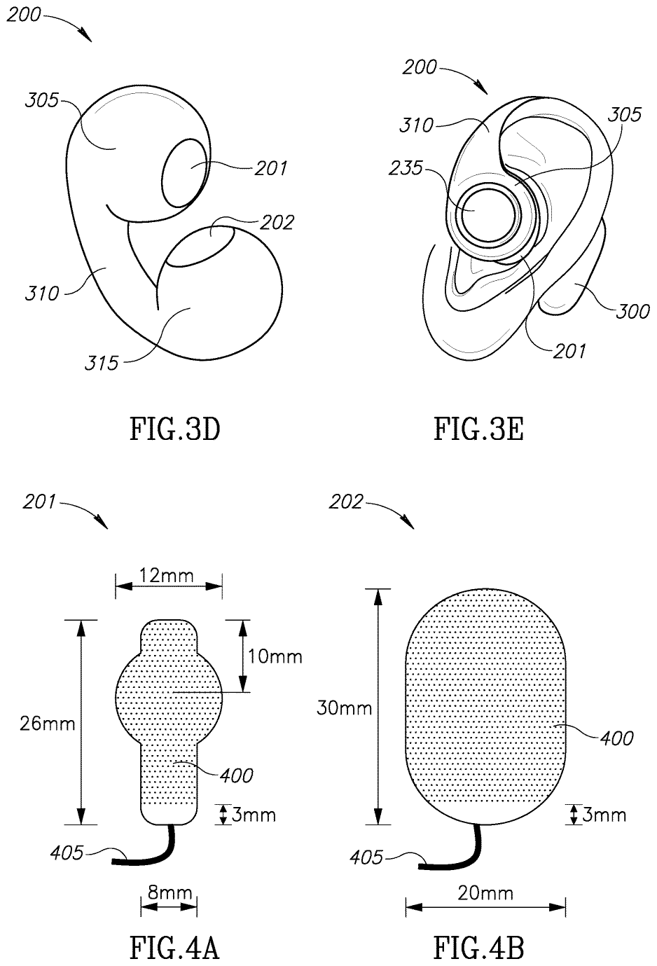

As shown in FIGS. 3A-3C, ear clip 300 is molded to facilitate positioning of ear clip 300 onto ear 10 to fit, thereby ensuring that the simulator portion does not exert any stresses on the ear 10. The structure of ear clip 300 thereby eliminates any problems caused by pressure produced by ear clip 300 positioned on ear 10. However, in some cases ear clip 300 may fall off from ear 10. A solution to the fall hazards can be either anatomic fitting of ear clip 300 to a personal ear structure of ear 10 or improving the adhesive properties of the electrode's adhesion gel 400 (FIGS. 4A-4B).

If the required stimulation power for continued use surpasses the capabilities of the power supplied by battery 245 (FIG. 2), system 200 can be connected to a charging headband (not shown). The charging headband can charge system 200 on a regular basis, such as every hour or two hours, thereby providing the necessary power for executing the stimulation activity of system 200. Alternatively, the charging headband can constantly charge battery 245 or replace the requirement for battery 245 in system 200.

In some embodiments, charging headband or any other external charging element can be utilized to charge system 200 through wireless charging, for example, through magnetic, electric, acoustic, light energies or the like.

The charging headband can be constructed to fit over ear clip 300 and transmit charging electricity to power supply 240 (FIG. 2). In some embodiments, the charging headband is firmly fitted over the head of a patient (not shown), potentially in several sizes to accommodate various patient head sizes. In some embodiments, the charging headband provides a solution for address potential sweating from the forehead or other body parts of the patient, for example, by the charging headband being firm but comfortable for the patient to wear.

In some embodiments, the charging headband can perform additional functions such as communication and conditioning circuitry. In some embodiments, ear clip 300 includes a power transfer circuitry on one side and the stimulation circuitry on the other. There is no need to pass on the stimulation parameters from the charging headband because the powering level and timing may precisely meet the needed stimulation in time and power level. Ear clip 300 facilitates providing the desired stimulation while adding function, weight, and volume to the charging headband.

In some embodiments, the ear clip 300 or the charging headband can be installed with photovoltaic cells (not shown) over the exposed ear clip 300 surfaces, which can charge battery 245 while the patient is in a well-lit environment. In some embodiments, such an enhancement can replace the charging circuitry or increase capabilities when there is a charging circuitry.

Auditorial stimulation in the human ear reception band and/or outside of this range is another function that can be achieved with ear clip 300. To improve the results, auditory stimulation can be initiated at specific moments before, during, or after the electrical stimulation. Because both stimulations target the brain, increasing the effectiveness of the combined stimulation can improve the desired results.

In some embodiments, ear clip 300 can be equipped with an accelerometer or other movement sensor to monitor the patient's status and trigger the stimulation as needed. For example, recognizing Parkinson's or epilepsy patients' seizures and activating or deactivating stimulation in response to reduce the effects of the seizure or involuntary movements. In some embodiments, a movement sensor can also facilitate detecting REM and non-REM periods of the sleeping rhythm. In other exemplary embodiments, the movement sensor can facilitate the system 200 recognizing the patient's activities, such as when the patient is walking, standing, or lying down, and triggering stimulation during one or more of these activities while not triggering stimulation during other activities.

In some embodiments, for example in FIGS. 3D and 3E, ear clip 300 incorporates an elastic clip 310 in the arm between the two stimulator sections that applies controlled force to maintain ear clip 300 in place rather than relying on the ear 10. The elastic clip 310 power limits can be 30, 60, or 100 milli Bar to provide adequate holding forces, and the maximum limit can be 400, 600, or 1,000 milli Bar to ensure tolerable pain for the patient. To prevent clip penetration into the skin of ear 10, the ear clip 300 force should be delivered in a wide enough surface area, at least 0.1, or 0.4 squared millimeter (mm2), on each side of ear 10. Although the ear clip power can inconvenience the patient, this notion is more dependable on that it can ensure that the ear clip 300 is kept in place, for example, while the patient is sleeping or running.

In some embodiments ear clip 300 can include alternative or additional stimulation modes, such as vibration and acoustical stimulation, such as sound or ultrasound, or the like. The additional stimulation may target specific ranges of stimulation frequencies that fit vagal activations.

With reference to FIGS. 4A-4B are schematic illustrations of active electrode 201 and return electrode 202 respectively, according to certain exemplary embodiments. The electrodes-active electrode 201 and return electrode 202, of system 200 are configured to be large enough to ensure low charge density during stimulation while compact enough to suit the stimulation sites: concha cavum and/or the concha cyma for the active electrode 201 and Eminence of the Concha for the return electrode 202. Each electrode-active electrode 201 and return electrode 202, can be tailored to fit precisely in its predetermined location. In some embodiments, active electrode 201 and return electrode 202 can be replaceable or disposable electrodes that may improve skin adherence and electrical impedance during stimulation.

In some embodiments, the active electrode 201 comprises an active area within a range of 50 to 1000 squared millimeters. In some other embodiments, the active electrode 201 includes an active area within a range of 100 to 600 squared millimeters. In some other embodiments, the active electrode 201 includes an active area within a range of 100 to 300 squared millimeters.

In some embodiments, the return electrode 202 comprises an active surface area within a range of 50 to 2000 squared millimeters. In some other embodiments, the active electrode 201 includes an active surface area within a range of 100 to 1000 squared millimeters. In some other embodiments, the active electrode 201 includes an active surface area within a range of 200 to 800 squared millimeters.

A disposable electrode should be physically and electrically connected to system 200, for example a wired pig tail connection 405 for electricity, which is accompanied by gel adhesion 400 to the stimulator on one side and the skin on the other. It is preferable to attach the electrode directly to the stimulator, which addresses both the electrical and physical connection. Among the technologies that can allow such instantaneous interactions are:

Electrically conductive hook & loop tape on both the stimulator and the electrode raises the height of the electrode in the small space by a few millimeters while allowing for an easy replacement technique and a strong physical and electrical connection.

In some embodiments, active electrode 201 and return electrode 202 can be click on replaceable electrodes with particular fitting in the stimulator circuitry 204, which can overcome these issues and requires a specially designed electrode.

In some embodiments, active electrode 201 and return electrode 202 can be fixed electrodes with a replaceable adhesion gel 400 on top, connected to the each electrode on one face and to the skin of the patient on the other.

In some embodiments, the stimulator circuitry can include a cooling element that cools the stimulation surface of active electrode 201 and return electrode 202. This may be used in a patient that has a very sensitive concha skin.

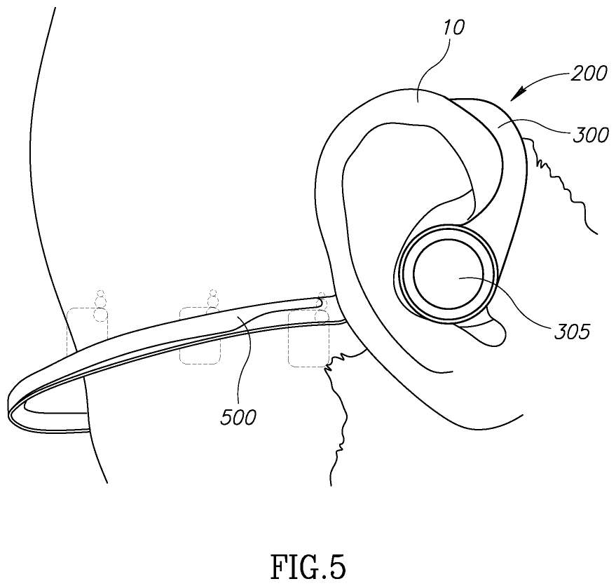

FIG. 5 shows a dual ear neurostimulator structure 500 and mounting, according to certain exemplary embodiments. The dual-ear stimulator 500 with a supporting arm 505 connecting the two ear devices 300 into a wireless single unit is another option for situations where both ears require stimulation. This single item may unify the electronic circuitries, such as the battery 245 (FIG. 2) being placed behind the left ear and the electrical circuitry for both ears being positioned behind the right ear. This approach can reduce clip pressure on each ear while replacing it with pressure applied by the connecting arm to keep the device firmly in place.

The connecting arm can be used to dissipate heat from the stimulating circuitry, moving it away from the patient's skin and spreading it over a broader region, among other things. This task can be performed with no moving parts by incorporating a heat pipe within the support arm.

In the context of some embodiments of the present disclosure, by way of example and without limiting, terms such as ‘operating’ or ‘executing’ imply also capabilities, such as ‘operable’ or ‘executable’, respectively. Conjugated terms such as, by way of example, ‘a thing property’ implies a property of the thing, unless otherwise clearly evident from the context thereof.

The terms ‘processor’ or ‘computer’, or system thereof, are used herein as ordinary context of the art, such as a general purpose processor or a micro-processor, RISC processor, or DSP, possibly comprising additional elements such as memory or communication ports. Optionally or additionally, the terms ‘processor’, ‘controller’, ‘computer’ or derivatives thereof denote an apparatus that is capable of carrying out a provided or an incorporated program and/or is capable of controlling and/or accessing data storage apparatus and/or other apparatus such as input and output ports. The terms ‘processor’, ‘controller’ or ‘computer’ denote also a plurality of processors or computers connected, and/or linked and/or otherwise communicating, possibly sharing one or more other resources such as a memory.

The terms ‘software’, ‘program’, ‘software procedure’ or ‘procedure’ or ‘software code’ or ‘code’ or ‘application’ may be used interchangeably according to the context thereof, and denote one or more instructions or directives or circuitry for performing a sequence of operations that generally represent an algorithm and/or other process or method. The program is stored in or on a medium such as RAM, ROM, or disk, or embedded in a circuitry accessible and executable by an apparatus such as a processor or other circuitry.

The processor and program may constitute the same apparatus, at least partially, such as an array of electronic gates, such as FPGA or ASIC, designed to perform a programmed sequence of operations, optionally comprising or linked with a processor or other circuitry.

The term computerized apparatus or a computerized system or a similar term denotes an apparatus comprising one or more processors operable or operating according to one or more programs.

As used herein, without limiting, a module represents a part of a system, such as a part of a program operating or interacting with one or more other parts on the same unit or on a different unit, or an electronic component or assembly for interacting with one or more other components.

As used herein, without limiting, a process represents a collection of operations for achieving a certain objective or an outcome.

As used herein, the term ‘server’ denotes a computerized apparatus providing data and/or operational service or services to one or more other apparatuses.

The term ‘configuring’ and/or ‘adapting’ for an objective, or a variation thereof, implies using at least a software and/or electronic circuit and/or auxiliary apparatus designed and/or implemented and/or operable or operative to achieve the objective.

A device storing and/or comprising a program and/or data constitutes an article of manufacture. Unless otherwise specified, the program and/or data are stored in or on a non-transitory medium.

In case electrical or electronic equipment is disclosed it is assumed that an appropriate power supply is used for the operation thereof.

The flowchart and block diagrams illustrate architecture, functionality or an operation of possible implementations of systems, methods and computer program products according to various embodiments of the present disclosed subject matter. In this regard, each block in the flowchart or block diagrams may represent a module, segment, or portion of program code, which comprises one or more executable instructions for implementing the specified logical function(s). It should also be noted that, in some alternative implementations, illustrated or described operations may occur in a different order or in combination or as concurrent operations instead of sequential operations to achieve the same or equivalent effect.

The corresponding structures, materials, acts, and equivalents of all means or step plus function elements in the claims below are intended to include any structure, material, or act for performing the function in combination with other claimed elements as specifically claimed. As used herein, the singular forms “a”, “an” and “the” are intended to include the plural forms as well, unless the context clearly indicates otherwise. It will be further understood that the terms “comprises” and/or “comprising” and/or “having” when used in this specification, specify the presence of stated features, integers, steps, operations, elements, and/or components, but do not preclude the presence or addition of one or more other features, integers, steps, operations, elements, components, and/or groups thereof.

As used herein the term “configuring” and/or ‘adapting’ for an objective, or a variation thereof, implies using materials and/or components in a manner designed for and/or implemented and/or operable or operative to achieve the objective.

Unless otherwise specified, the terms ‘substantially’, ‘about’ and/or ‘close’ with respect to a magnitude or a numerical value implies within an inclusive range of −10% to +10% of the respective magnitude or value. Unless otherwise specified, the terms ‘substantially’, ‘about’ and/or ‘close’ with respect to a dimension or extent, such as length, implies within an inclusive range of −10% to +10% of the respective dimension or extent.

Unless otherwise specified, the terms ‘about’ or ‘close’ imply at or in a region of, or close to a location or a part of an object relative to other parts or regions of the object.

When a range of values is recited, it is merely for convenience or brevity and includes all the possible sub-ranges as well as individual numerical values within and about the boundary of that range. Any numeric value, unless otherwise specified, includes also practical close values enabling an embodiment or a method, and integral values do not exclude fractional values. A sub-range values and practical close values should be considered as specifically disclosed values. As used herein, ellipsis ( . . . ) between two entities or values denotes an inclusive range of entities or values, respectively. For example, A . . . Z implies all the letters from A to Z, inclusively.

The terminology used herein should not be understood as limiting, unless otherwise specified, and is for the purpose of describing particular embodiments only and is not intended to be limiting of the disclosed subject matter. While certain embodiments of the disclosed subject matter have been illustrated and described, it will be clear that the disclosure is not limited to the embodiments described herein. Numerous modifications, changes, variations, substitutions and equivalents are not precluded.

Terms in the claims that follow should be interpreted, without limiting, as characterized or described in the specification.

1. A noninvasive auricular neurostimulator comprising:
a stimulation device for activating an auricular branch of the Vagus nerve, said stimulation device comprising:
an active electrode facing a concha cavum of the ear; and,
a return electrode facing an Eminence of the concha, or the retro auricular sulcus.

2. The noninvasive auricular neurostimulator of claim 1, further comprising an elastic clip.

3. The noninvasive auricular neurostimulator of claim 2, wherein said elastic clip requires pressure power within a range of 10 to 500 milliBars.

4. The noninvasive auricular neurostimulator according to claim 1, further comprising a communication system.

5. The noninvasive auricular neurostimulator according to claim 4, wherein said communication system is wireless.

6. The noninvasive auricular neurostimulator according to claim 1, wherein said return electrode comprises an active area within a range of 10 to 1000 squared millimeters.

7. The noninvasive auricular neurostimulator according to claim 1, wherein said active electrode comprises an active area within a range of 50 to 400 squared millimeters.

8. The noninvasive auricular neurostimulator according to claim 1, wherein said active electrode and said return electrode are disposable in part or in full.

9. The noninvasive auricular neurostimulator according to claim 1, wherein said active electrode and said return electrode each comprise a disposable electrically conductive gel leaf.

10. The noninvasive auricular neurostimulator according to claim 1, wherein a stimulation activity is synchronized to the physiological states of a patient.

11. The noninvasive auricular neurostimulator according to claim 1, wherein said stimulation device is configured to treat neurological disorders.

12. The noninvasive auricular neurostimulator according to claim 11, wherein the neurological disorder is Alzheimer disease.

13. The noninvasive auricular neurostimulator according to claim 1, further comprising two stimulation sites, one site on the left ear concha and the other site on the right ear concha.

14. The noninvasive auricular neurostimulator according to claim 13, wherein the two stimulation sites are connected by an elastic bar that provides pressure at an electrode interface within the concha.

15. The noninvasive auricular neurostimulator according to claim 14, wherein the one or more electrode on one or 2 ears are configured for sensing Electroencephalography (“EEG”) or Electrocardiogram (ECG) signals.

16. The noninvasive auricular neurostimulator according to claim 1, further comprising an additional module configured for acoustic stimulation or mechanical vibration stimulation.

17. The noninvasive auricular neurostimulator according to claim 1, further comprising an additional headband configured to directly charge said auricular neurostimulator device.

18. The noninvasive auricular neurostimulator of claim 1, wherein said electrodes are placed in a way that the electrodes are pushed toward the concha skin or having an adhesive gel that enables contact with the skin.

19. The noninvasive auricular neurostimulator of claim 1, wherein said stimulation device is positioned adjacent to the ear and connected with cables to said active electrode.

20-23. (canceled)
